(19) United States
(12) Patent Application Publication
(10) Pub. No.: US 2022/0370682 A1
(43) Pub. Date: Nov. 24, 2022

(54) BIOACTIVE SCAFFOLD AUGMENTATION FOR ACL RECONSTRUCTION

(71) Applicants: Smith & Nephew, Inc., Memphis, TN (US); Smith & Nephew Orthopaedics AG, Zug (CH); Smith & Nephew Asia Pacific Pte. Limited, SINGAPORE (SG)

(72) Inventors: Stephen A. Santangelo, Sturbridge, MA (US); David Alexander Yeung, Andover, MA (US); Nicholas John Cotton, Westborough, MA (US)

(73) Assignees: Smith & Nephew, Inc., Memphis, TN (US); Smith & Nephew Orthopaedics AG, Zug (CH); Smith & Nephew Asia Pacific Pte. Limited, SINGAPORE (SG)

(21) Appl. No.: 17/875,114

(22) Filed: Jul. 27, 2022

Related U.S. Application Data
(63) Continuation of application No. PCT/US2021/018207, filed on Feb. 16, 2021.
(60) Provisional application No. 62/977,871, filed on Feb. 18, 2020.

(51) Int. Cl. A61L 27/24; A61L 27/56; A61F 2/08

(52) U.S. Cl. CPC A61L 27/24; A61L 27/56; A61F 2/0811; A61F 2002/087; A61L 2430/10; A61F 2002/0882; A61F 2002/0852

(57) ABSTRACT

A bioactive scaffold for use in reconstruction of an anterior cruciate ligament of a patient, may include a tissue matrix configured to wrap around a graft configured to extend from a first bone of a joint to a second bone of the joint. The tissue matrix may include collagen type I. The tissue matrix may include a first plurality of apertures extending through the tissue matrix along a first edge of the tissue matrix and a second plurality of apertures extending through the tissue matrix along a second edge of the tissue matrix. At least one filament may be configured to be laced through the first plurality of apertures and the second plurality of apertures and draw the first edge of the tissue matrix toward the second edge of the tissue matrix when the at least one filament is subjected to tension thereby closing the tissue matrix around the graft.

### CROSS REFERENCE TO RELATED APPLICATIONS

This application is a continuation of International Application No. PCT/US 2021/018207, filed Feb. 16, 2021, which claims the benefit of U.S. Provisional Patent Application Ser. No. 62/977,871 filed on Feb. 18, 2020, the disclosures of which are incorporated herein by reference.

### TECHNICAL FIELD

The present disclosure pertains to medical devices, and methods for manufacturing and/or using medical devices. More particularly, the present disclosure pertains to a bioactive scaffold for use in anterior cruciate ligament (ACL) reconstruction surgery.

### BACKGROUND

Injuries to the anterior cruciate ligament (ACL) and/or other ligaments may range from minor (partial tear) to catastrophic (rupture). For some patients, the method of treatment may depend on the severity of the injury. ACL reconstruction surgery may be appropriate where the injury is so severe that injured ACL must be replaced. ACL reconstruction surgery may involve preparing a graft, which may include harvesting a tendon, ligament, or other tissue from another location within the patient's body, and implanting the graft in place of the injured ACL. Similar procedures may be used for other injured ligaments, including but not limited to the lateral collateral ligament (LCL), the posterior cruciate ligament (PCL), the medial collateral ligament (MCL), etc. The healing time for ACL reconstruction is significant, and the reconstructed ACL may be vulnerable to injury during the healing process. There is an ongoing need to provide alternative medical devices as well as alternative methods for manufacturing and using medical devices for use in ACL reconstruction.

### SUMMARY

In one example, a bioactive scaffold for use in reconstruction of an anterior cruciate ligament of a patient may comprise a tissue matrix configured to wrap around a graft, the graft being configured to extend from a first bone of a joint to a second bone of the joint. The tissue matrix may include collagen type I. The tissue matrix may include a first plurality of apertures extending through the tissue matrix along a first edge of the tissue matrix and a second plurality of apertures extending through the tissue matrix along a second edge of the tissue matrix. At least one filament may be configured to be laced through the first plurality of apertures and the second plurality of apertures and draw the first edge of the tissue matrix toward the second edge of the tissue matrix when the at least one filament is subjected to tension thereby closing the tissue matrix around the graft.

In addition or alternatively to any example described herein, after the at least one filament is subjected to tension to close the tissue matrix around the graft, the tissue matrix applies a compressive force to the graft.

In addition or alternatively to any example described herein, the first edge of the tissue matrix is configured to abut the second edge of the tissue matrix when the at least one filament is subjected to tension to close the tissue matrix around the graft.

In addition or alternatively to any example described herein, the first edge of the tissue matrix is configured to circumferentially overlap the second edge of the tissue matrix when the at least one filament is subjected to tension to close the tissue matrix around the graft.

In addition or alternatively to any example described herein, the first plurality of apertures is aligned with the second plurality of apertures.

In addition or alternatively to any example described herein, the first plurality of apertures is disposed opposite the second plurality of apertures relative to the first edge of the tissue matrix.

In addition or alternatively to any example described herein, the at least one filament is configured to be laced through the first plurality of apertures and the second plurality of apertures in an alternating fashion.

In addition or alternatively to any example described herein, a first portion of the graft is configured to extend into a first hole formed in the first bone.

In addition or alternatively to any example described herein, a first portion of the tissue matrix is configured to extend into the first hole formed in the first bone.

In addition or alternatively to any example described herein, a second portion of the graft is configured to extend into a second hole formed in the second bone.

In addition or alternatively to any example described herein, a second portion of the tissue matrix is configured to extend into the second hole formed in the second bone.

In addition or alternatively to any example described herein, a bioactive scaffold for use in reconstruction of an anterior cruciate ligament of a patient may comprise a tissue matrix configured to wrap around a graft, the graft being configured to extend from a first bone of a joint to a second bone of the joint. The tissue matrix may include collagen type I. The tissue matrix may be wrapped helically around the graft.

In addition or alternatively to any example described herein, after wrapping the tissue matrix around the graft, the tissue matrix is in direct contact with the graft from a first location adjacent the first bone to a second location adjacent the second bone.

In addition or alternatively to any example described herein, a first edge of the tissue matrix is configured to abut a second edge of the tissue matrix after wrapping the tissue matrix around the graft.

In addition or alternatively to any example described herein, a first edge of the tissue matrix is configured to circumferentially overlap a second edge of the tissue matrix after wrapping the tissue matrix around the graft.

In addition or alternatively to any example described herein, the tissue matrix is configured to retain its shape and position relative to the graft after being helically wrapped around the graft.

In addition or alternatively to any example described herein, a bioactive scaffold for use in reconstruction of an anterior cruciate ligament of a patient may comprise a tissue matrix configured to surround a graft, the graft being configured to extend from a first bone of a joint to a second bone of the joint. The graft may include a first tether extending axially from the graft, the first tether being configured to secure the graft to the first bone. The tissue matrix may include collagen type I. The tissue matrix may include a first orifice proximate a first end of the graft, the first tether extending axially through the first orifice. A cross-sectional area of the first orifice may be less than a cross-sectional area of the graft proximate the first end of the graft. The cross-sectional area of the first orifice may be at least twice as great as a cross-sectional area of the first tether. The tissue matrix may be configured to span a gap between the first bone and the second bone.

In addition or alternatively to any example described herein, the first tether is configured to extend into a first hole formed in the first bone to a first anchoring member.

In addition or alternatively to any example described herein, the first anchoring member is configured to rest against an outside surface of the first bone when engaged with the first tether.

In addition or alternatively to any example described herein, the tissue matrix has a thickness of 3 millimeters or less.

The above summary of some embodiments, aspects, and/or examples is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates an anterior view of a knee having a normal ACL;

FIG. 2 illustrates an anterior view of a knee having a torn ACL;

FIG. 3 illustrates aspects of a knee undergoing ACL reconstruction surgery;

FIG. 4 illustrates aspects of an example bioactive scaffold for use in ACL reconstruction surgery;

FIGS. 5A and 5B illustrate aspects of the bioactive scaffold of FIG. 4 in use during ACL reconstruction surgery;

FIGS. 6A and 6B illustrate aspects of the bioactive scaffold of FIG. 4 in use during ACL reconstruction surgery;

FIG. 7 illustrates aspects of an example bioactive scaffold for use in ACL reconstruction surgery;

FIG. 8 illustrates aspects of the bioactive scaffold of FIG. 7 in use during ACL reconstruction surgery;

FIG. 9 illustrates aspects of the bioactive scaffolds of FIGS. 4 and 7 in use during ACL reconstruction surgery;

FIG. 10 illustrates aspects of the bioactive scaffolds of FIGS. 4 and 7 in use during ACL reconstruction surgery;

FIG. 11 illustrates aspects of an example bioactive scaffold for use in ACL reconstruction surgery;

FIG. 12 illustrates aspects of the bioactive scaffold of FIG. 11 in use during ACL reconstruction surgery;

FIG. 13 illustrates aspects of an alternative configuration of the bioactive scaffold of FIGS. 11-12; and

FIG. 14 illustrates aspects of an alternative configuration of the bioactive scaffold of FIGS. 11-12.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosed invention are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The figures illustrate selected components and/or arrangements of a bioactive scaffold. It should be noted that in any given figure, some features of the bioactive scaffold may not be shown, or may be shown schematically, for simplicity. Additional details regarding some of the elements of the bioactive scaffold may be illustrated in other figures in greater detail. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to features or elements may be equally referred to all instances and quantities beyond one of said feature or element. As such, it will be understood that the following discussion may apply equally to any and/or all of the elements for which there are more than one within the bioactive scaffold, unless explicitly stated to the contrary. Additionally, not all instances of some elements or features may be shown in each figure for clarity.

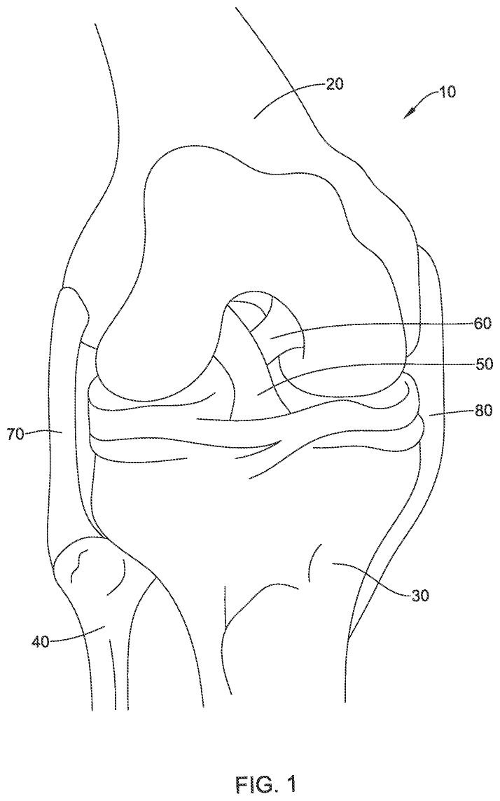

FIG. 1 illustrates an anterior view of a knee 10 of a human patient showing selected features of the anatomy of the knee 10. Bones associated with the knee 10 may include the femur 20, the tibia 30, and the fibula 40. The patella (kneecap) is omitted for clarity. The knee 10 may include a plurality of ligaments connecting the bones to stabilize the knee 10. The ligaments associated with the knee 10 may include the anterior cruciate ligament 50

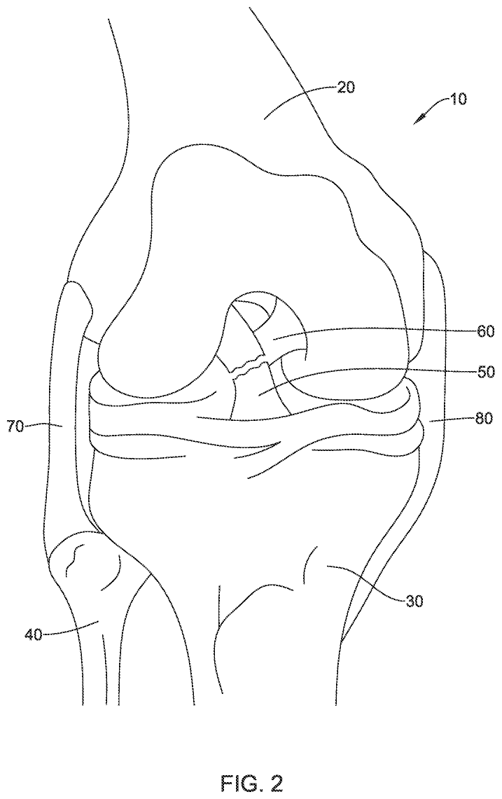

(ACL), the posterior cruciate ligament 60 (PCL), the lateral collateral ligament 70 (LCL), and the medial collateral ligament 80 (MCL). FIG. 1 illustrates an anterior cruciate ligament 50 in a substantially “normal” condition (e.g., uninjured, intact, untorn, etc.). FIG. 2 illustrates the knee 10 having the anterior cruciate ligament 50 in an injured condition. For example, the anterior cruciate ligament 50 may be partially torn or completely torn (ruptured). For the purpose of illustration only, the anterior cruciate ligament 50 is shown completely torn (ruptured), which normally required ACL reconstruction surgery. However, ACL reconstruction surgery may also be performed in patients having a partially torn ACL.

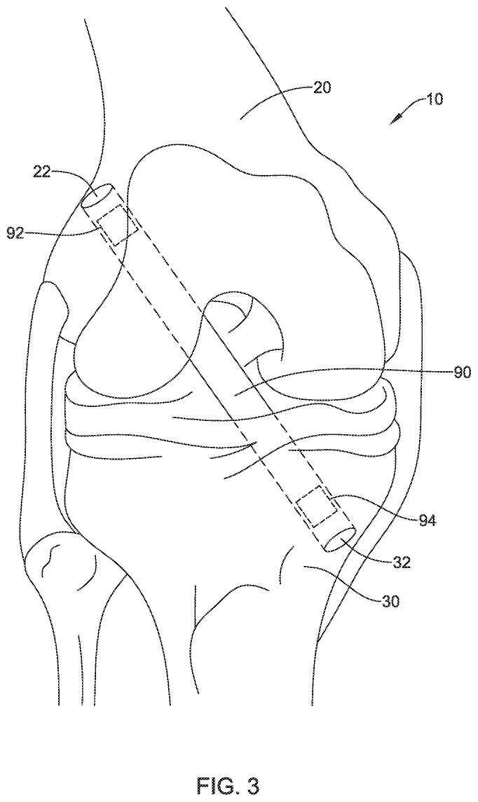

FIG. 3 illustrates the knee 10 partially through the process of ACL reconstruction. In some embodiments, a hole may be drilled into one or more bones at or near the site of an injured anterior cruciate ligament with conventional surgical techniques using conventional surgical drills and drill guides. For example, a first hole 22 may be formed in a first bone of the knee 10 (e.g., the femur 20) and a second hole 32 may be formed in a second bone of the knee 10 (e.g., the tibia 30), as seen in FIG. 3. During the procedure, the injured anterior cruciate ligament 50 may be removed according to known techniques. A graft 90 may then be prepared and mounted in the first hole 22 and/or the second hole 32, as discussed in more detail herein, and the graft 90 may be secured to the first bone (e.g., the femur 20) with a first anchor member 92 and/or the second bone (e.g., the tibia 30) with a second anchor member 94. In at least some embodiments, conventional securement techniques may be used. For example, the first anchor member 92 and/or the second anchor member 94 may be a threaded bone anchor or other suitable means of securing the graft 90 to the first bone and/or the second bone.

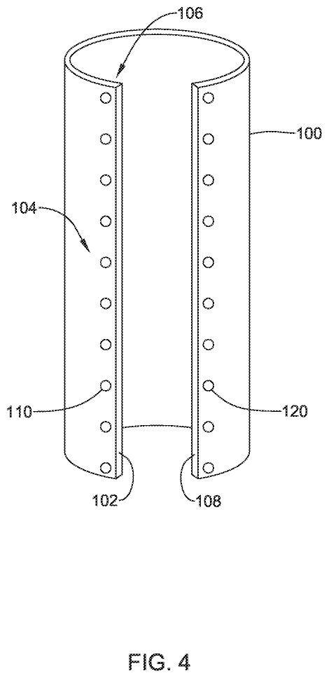

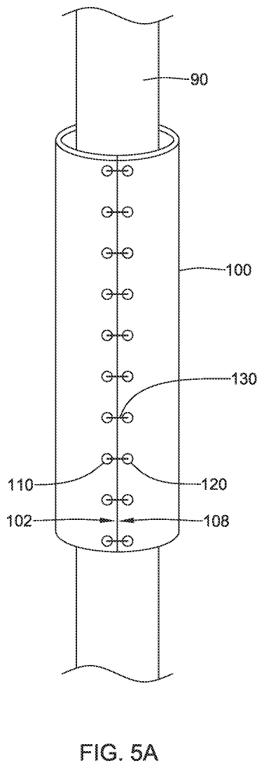

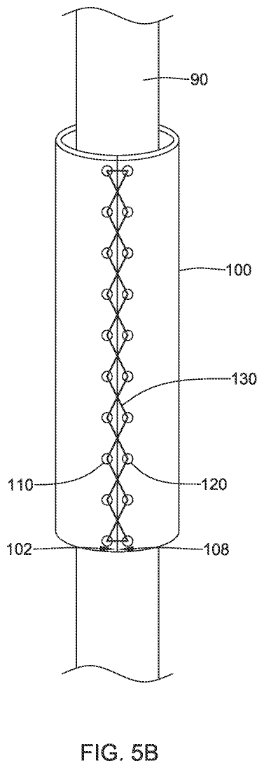

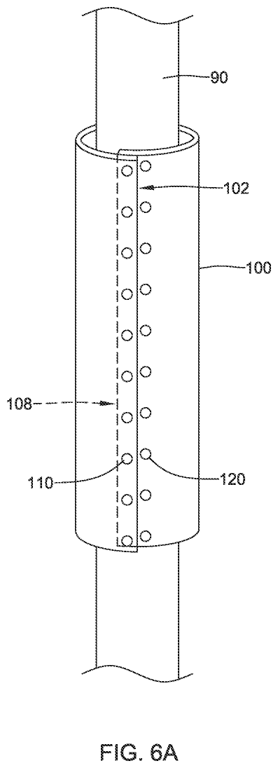

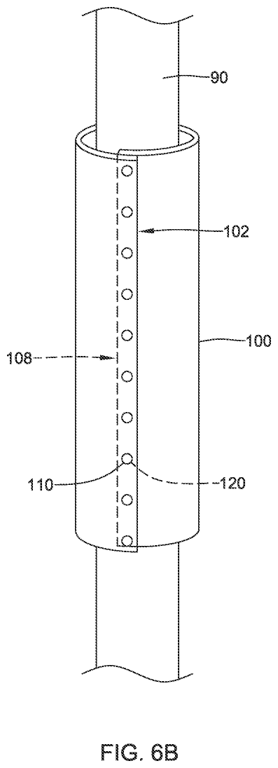

FIG. 4 illustrates a bioactive scaffold for use in reconstruction of an anterior cruciate ligament of a patient. The bioactive scaffold may comprise a tissue matrix 100 configured to wrap around the graft 90 (e.g., FIGS. 5A-6B), the graft being configured to extend from a first bone (e.g., the femur 20) of a joint (e.g., the knee 10) to a second bone (e.g., the tibia 30) of the joint. In at least some embodiments, the graft 90 may be an autograft (e.g., a tendon, ligament, or other tissue) harvested from the patient. In some embodiments, the graft 90 may be an allograft (e.g., a ligament harvested from a cadaver) or a xenograft (e.g., connective tissue harvested from animal sources). In some embodiments, the bioactive scaffold and/or the tissue matrix 100 may be wrapped around the graft 90 before the graft 90 is implanted into the joint. In some other embodiments, the bioactive scaffold and/or the tissue matrix 100 may be wrapped around the graft 90 after the graft 90 is implanted into the joint.

In some embodiments, the tissue matrix 100 may include and/or be formed from a biocompatible material such as collagen. Preferably, the tissue matrix 100 includes collagen type I, although other forms of collagen (e.g., types II, III, IV, V, IX, or X) may be used and/or may be included and/or mixed with collagen type I. In some embodiments, the tissue matrix 100 may have a thickness of about 5 millimeters or less, about 4 millimeters or less, about 3 millimeters or less, about 2 millimeters or less, or another suitable thickness. Preferably, the tissue matrix 100 has a thickness of about 3 millimeters or less. In some embodiments, the bioactive scaffold and/or the tissue matrix 100 may include bioactive components configured to stimulate a healing response within the patient. In some embodiments, the composition of the bioactive scaffold and/or the tissue matrix 100 may be varied and/or optimized to promote a desired healing response in each anatomical portion of the graft 90. In some embodiments, the bioactive scaffold and/or the tissue matrix 100 may be coated and/or impregnated with a therapeutic substance configured to promote or inhibit a particular response from the patient, the graft 90, and/or the tissue matrix 100.

In some embodiments, the tissue matrix 100 may include a first plurality of apertures 110 extending through the tissue matrix 100 along a first edge 102 of the tissue matrix 100. In at least some embodiments, the first plurality of apertures 110 may extend completely through a thickness of the tissue matrix 100. In some embodiments, the first plurality of apertures 110 may extend though the tissue matrix 100 orthogonally to a first surface 104 and/or a second surface 106 opposing the first surface 104. In some embodiments, the tissue matrix 100 may include a second plurality of apertures 120 extending through the tissue matrix 100 along a second edge 108 of the tissue matrix 100. In at least some embodiments, the second plurality of apertures 120 may extend completely through the thickness of the tissue matrix 100. In some embodiments, the second plurality of apertures 120 may extend though the tissue matrix 100 orthogonally to the first surface 104 and/or the second surface 106 opposing the first surface 104.

In some embodiments, the bioactive scaffold may include at least one filament 130 configured to be laced through the first plurality of apertures 110 and the second plurality of apertures 120. In some embodiments, the at least one filament 130 may include one filament, two filaments, three filaments, four filaments, five filaments, a plurality of filaments, or another suitable number of filaments. The at least one filament 130 may be configured to draw the first edge 102 of the tissue matrix 100 toward the second edge 108 of the tissue matrix 100 when the at least one filament 130 is subjected to tension, thereby closing the tissue matrix 100 around the graft 90. In some embodiments, after the at least one filament 130 is subjected to tension to close the tissue matrix 100 around the graft 90, the tissue matrix 100 applies a compressive force to the graft 90. In some embodiments, after the at least one filament 130 is subjected to tension to close the tissue matrix 100 around the graft 90, the tissue matrix 100 may limit contact between the graft 90 and synovial fluid within the joint (e.g., the knee 10).

As shown in FIGS. 5A and 5B, the at least one filament 130 may be laced through the first plurality of apertures 110 and the second plurality of apertures 120 in one or more of several arrangements. In one example, the at least one filament 130 may be configured to be laced through one of the first plurality of apertures 110 and one of the second plurality of apertures 120 positioned adjacent and/or proximate to one another. In some embodiments, a different filament may be laced through each adjacent and/or opposing pair of apertures. In some embodiments, the at least one filament 130 may be configured to extend laterally across the first edge 102 and the second edge 108 between the first plurality of apertures 110 and the second plurality of apertures 120. In some embodiments, the at least one filament 130 may be configured to extend parallel to the first plurality of apertures 110 and the second plurality of apertures 120. In some embodiments, the at least one filament 130 may be configured to extend helically through the first plurality of apertures 110 and the second plurality of apertures 120. In some embodiments, the at least one filament 130 may be configured to be laced through the first plurality of apertures 110 and the second plurality of apertures 120 in an alternating fashion. For example, the at least one filament 130 may be configured to extend in a zigzag pattern between the first plurality of apertures 110 and the second plurality of apertures 120. Other arrangements are also contemplated. In some embodiments, combinations of these arrangements may be used.

As shown in FIGS. 5A and 5B for example, in some embodiments, the first edge 102 of the tissue matrix 100 may be configured to be positioned immediately adjacent the second edge 108 of the tissue matrix 100 when the at least one filament 130 is subjected to tension to close the tissue matrix 100 around the graft 90. In some embodiments, the first edge 102 of the tissue matrix 100 may be configured to contact the second edge 108 of the tissue matrix 100 when the at least one filament 130 is subjected to tension to close the tissue matrix 100 around the graft 90. In some embodiments, the first edge 102 of the tissue matrix 100 may be configured to abut the second edge 108 of the tissue matrix 100 when the at least one filament 130 is subjected to tension to close the tissue matrix 100 around the graft 90.

In some embodiments, the first edge 102 of the tissue matrix 100 may be configured to circumferentially overlap the second edge 108 of the tissue matrix 100 when the at least one filament 130 (not shown) is subjected to tension to close the tissue matrix 100 around the graft 90, as seen in FIGS. 6A and 6B. For example, in some embodiments, the tissue matrix 100 may be configured to be positioned such that the first edge 102 of the tissue matrix 100 is laterally and/or circumferentially offset from the second edge 108 of the tissue matrix 100 when the at least one filament 130 (not shown) is subjected to tension to close the tissue matrix 100 around the graft 90.

In some embodiments, the first plurality of apertures 110 may be aligned with the second plurality of apertures 120 when the first edge 102 of the tissue matrix 100 is drawn toward the second edge 108 of the tissue matrix 100. In some embodiments, the first plurality of apertures 110 may be disposed opposite the second plurality of apertures 120 relative to the first edge 102 of the tissue matrix 100 when the first edge 102 of the tissue matrix 100 is drawn toward the second edge 108 of the tissue matrix 100. In some embodiments, a center and/or a central axis of each of the first plurality of apertures 110 may be disposed opposite a center and/or a central axis of each of the second plurality of apertures 120 relative to the first edge 102 of the tissue matrix 100 when the first edge 102 of the tissue matrix 100 is drawn toward the second edge 108 of the tissue matrix 100. For example, in some embodiments, the first plurality of apertures 110 may mirror the second plurality of apertures 120 about the first edge 102 of the tissue matrix 100 when the first edge 102 of the tissue matrix 100 is drawn toward the second edge 108 of the tissue matrix 100. In some embodiments, a center and/or a central axis of each of the first plurality of apertures 110 may be aligned with a corresponding center and/or a central axis of each of the second plurality of apertures 120 when the first edge 102 of the tissue matrix 100 is drawn toward the second edge 108 of the tissue matrix 100.

In some embodiments, a bioactive scaffold for use in reconstruction of an anterior cruciate ligament of a patient may comprise a tissue matrix 200 configured to wrap around a graft 90, the graft 90 being configured to extend from a first bone (e.g., the femur 20) of a joint (e.g., the knee 10) to a second bone (e.g., the tibia 30) of the joint. In some embodiments, the bioactive scaffold and/or the tissue matrix 200 may be wrapped around the graft 90 before the graft 90 is implanted into the joint. In some other embodiments, the bioactive scaffold and/or the tissue matrix 200 may be wrapped around the graft 90 after the graft 90 is implanted into the joint.

In some embodiments, the tissue matrix 200 may include and/or be formed from a biocompatible material such as collagen. Preferably, the tissue matrix 200 includes collagen type I, although other forms of collagen (e.g., types II, III, IV, V, IX, or X) may be used and/or may be included and/or mixed with collagen type I. In some embodiments, the tissue matrix 200 may have a thickness of about 5 millimeters or less, about 4 millimeters or less, about 3 millimeters or less, about 2 millimeters or less, or another suitable thickness. Preferably, the tissue matrix 200 has a thickness of about 3 millimeters or less. In some embodiments, the bioactive scaffold and/or the tissue matrix 200 may include bioactive components configured to stimulate a healing response within the patient. In some embodiments, the composition of the bioactive scaffold and/or the tissue matrix 200 may be varied and/or optimized to promote a desired healing response in each anatomical portion of the graft 90. In some embodiments, the bioactive scaffold and/or the tissue matrix 200 may be coated and/or impregnated with a therapeutic substance configured to promote or inhibit a particular response from the patient, the graft 90, and/or the tissue matrix 200.

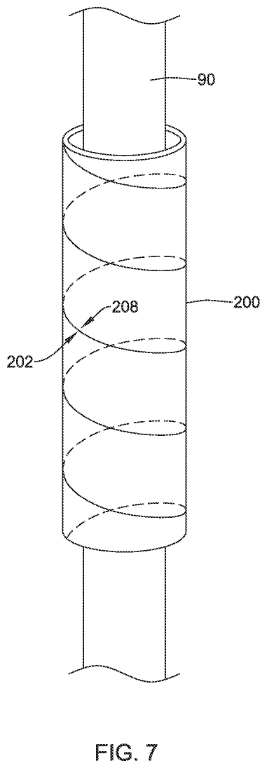

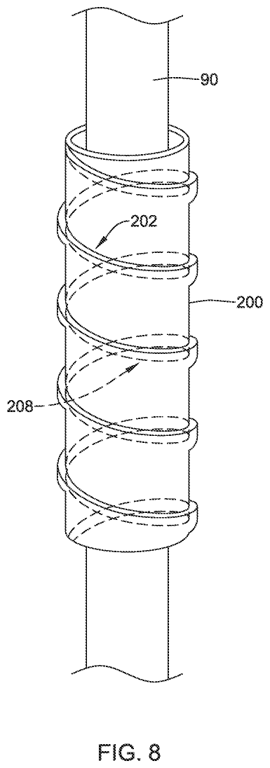

In some embodiments, the tissue matrix 200 may be configured to be wrapped helically around the graft 90, as seen in FIGS. 7 and 8. In some embodiments, the tissue matrix 200 may be configured to retain its shape and position relative to the graft 90 after being helically wrapped around the graft 90. In one non-limiting example, portions of the tissue matrix 200 may be secured and/or bonded to other portions of the tissue matrix 200 when and/or after the tissue matrix 200 is wrapped helically around the graft 90. In some embodiments, after wrapping the tissue matrix 200 around the graft 90, the tissue matrix 200 may limit contact between the graft 90 and synovial fluid within the joint (e.g., the knee 10).

In some embodiments, a first edge 202 of the tissue matrix 200 may be configured to be positioned immediately adjacent the second edge 208 of the tissue matrix 200 when and/or after the tissue matrix 200 is wrapped helically around the graft 90, as seen in FIG. 7. In some embodiments, the first edge 202 of the tissue matrix 200 may be configured to contact the second edge 208 of the tissue matrix 200 when and/or after the tissue matrix 200 is wrapped helically around the graft 90. In some embodiments, the first edge 202 of the tissue matrix 200 may be configured to abut the second edge 208 of the tissue matrix 200 when and/or after the tissue matrix 200 is wrapped helically around the graft 90.

In some embodiments, the first edge 202 of the tissue matrix 200 may be configured to circumferentially and/or longitudinally overlap the second edge 208 of the tissue matrix 200 when and/or after the tissue matrix 200 is wrapped helically around the graft 90, as seen in FIG. 8. For example, in some embodiments, the tissue matrix 200 may be configured to be positioned such that the first edge 202 of the tissue matrix 200 is circumferentially and/or longitudinally offset from the second edge 208 of the tissue matrix 200 when and/or after the tissue matrix 200 is wrapped helically around the graft 90. In some embodiments, the first edge 202 of the tissue matrix 200 may be configured to circumferentially and/or longitudinally overlap the second edge 208 of the tissue matrix 200 by at least a thickness of the tissue matrix 200 when and/or after the tissue matrix 200 is wrapped helically around the graft 90. In some embodiments, the first edge 202 of the tissue matrix 200 may be configured to circumferentially and/or longitudinally overlap the second edge 208 of the tissue matrix 200 by two, three, four, five, or more times the thickness of the tissue matrix 200 when and/or after the tissue matrix 200 is wrapped helically around the graft 90.

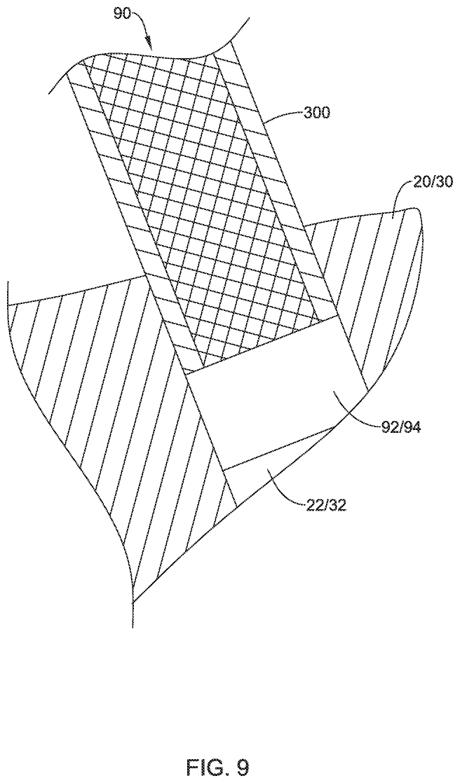

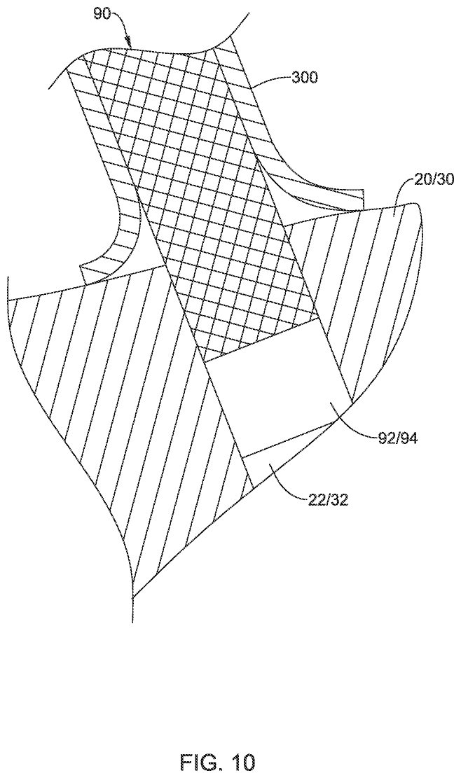

FIGS. 9 and 10 illustrate features of a tissue matrix 300 used in reconstruction of an anterior cruciate ligament of a patient along with the graft 90. In some embodiments, the tissue matrix 300 may be the tissue matrix 100 or the tissue matrix 200. As such, any and/or all aspects and/or features of the tissue matrix 300 may be applied to and/or with respect to the tissue matrix 100 or the tissue matrix 200, and these elements and/or reference numbers may be used and/or applied interchangeably herein.

In some embodiments, a first portion of the graft 90 may be configured to extend into the first hole 22 formed in the first bone 20. In some embodiments, a second portion of the graft 90 may be configured to extend into the second hole 32 of the second bone 30. In the interest of clarity, only one hole and one bone are illustrated in FIGS. 9 and 10, but the skilled artisan will recognize that the same general configuration may apply to both the first bone 20 and the second bone 30, as well as first and second opposing ends of the graft 90. In some embodiments, after wrapping the tissue matrix 300 around the graft 90, the tissue matrix 300 may be in direct contact with the graft 90 from a first location adjacent the first bone 20 to a second location adjacent to the second bone 30. In some embodiments, the first and second ends of the graft 90 may be secured to the first and second bones of the joint, respectively, using one or more securement means known in the art. In one example, the first end of the graft 90 may be secured to the first bone 20 using the first anchor member 92 (e.g., a bone screw, a nail, etc.) and/or the second end of the graft 90 may be secured to the second bone 30 using the second anchor member 94 (e.g., a bone screw, a nail, etc.). In another example, the first end and/or the second end of the graft 90 may be secured to the first bone and/or the second bone using an adhesive or bonding agent. In another example, the first end and/or the second end of the graft 90 may be secured to the first bone and/or the second bone using other mechanical fixation means, including but not limited to tethers, filaments, knots, etc. Other configurations, as well as combinations thereof, are also contemplated.

In some embodiments, a first portion of the tissue matrix 300 may be configured to extend into the first hole 22 formed in the first bone 20, as shown in FIG. 9. In some embodiments, the first portion of the tissue matrix 300 may extend along the graft 90 to the first anchor member 92. In one example, the first portion of the tissue matrix 300 may abut the first anchor member 92. Similarly, in some embodiments, a second portion of the tissue matrix 300 may be configured to extend into the second hole 32 formed in the second bone 30. In some embodiments, the second portion of the tissue matrix 300 may extend along the graft 90 to the second anchor member 94. In one example, the second portion of the tissue matrix 300 may abut the second anchor member 94. A third portion of the tissue matrix 300 may extend longitudinally along the graft 90 between the first portion of the tissue matrix 300 and the second portion of the tissue matrix 300.

In some embodiments, the first portion of the tissue matrix 300 may be configured to extend radially outward from the graft 90 and drape over the first bone 20 at and/or proximate the first hole 22, as shown in FIG. 10. In some embodiments, the first portion of the tissue matrix 300 may rest against an outside surface of the first bone 20 after the graft 90 and the tissue matrix 300 have been implanted into the patient's knee. In at least some embodiments, none of the tissue matrix 300 may extend into the first hole 22 formed in the first bone 20. In some embodiments, the second portion of the tissue matrix 300 may be configured to extend radially outward from the graft 90 and drape over the second bone 30 at and/or proximate the second hole 32. In some embodiments, the second portion of the tissue matrix 300 may rest against an outside surface of the second bone 30 after the graft 90 and the tissue matrix 300 have been implanted into the patient's knee. In at least some embodiments, none of the tissue matrix 300 may extend into the second hole 32 formed in the second bone 30.

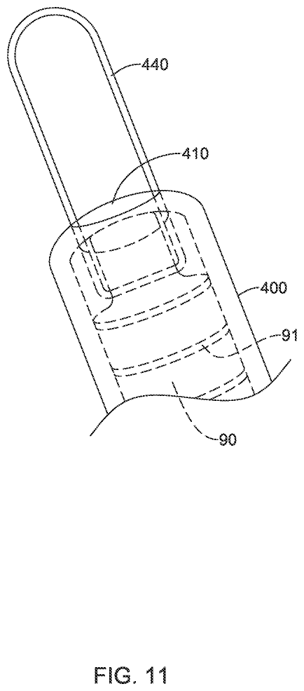

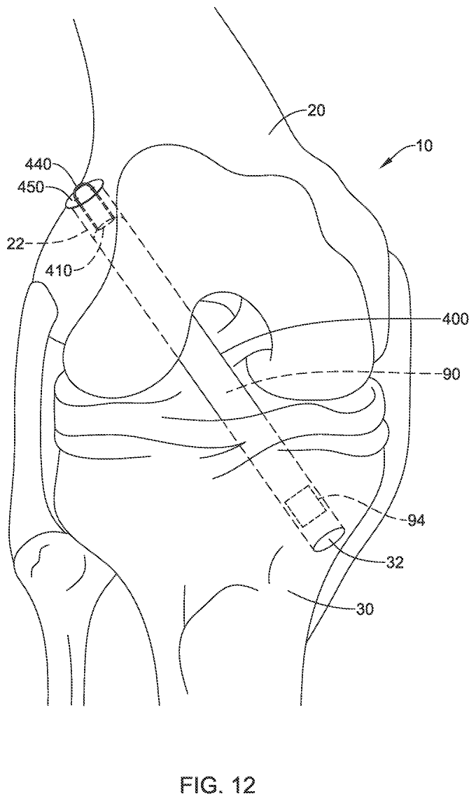

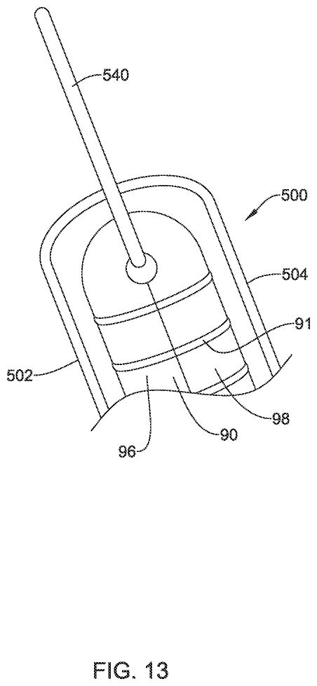

In an alternative embodiment illustrated in FIGS. 11 and 12, a bioactive scaffold for use in reconstruction of an anterior cruciate ligament of a patient may comprise a tissue matrix 400 configured to surround a graft 90, the graft 90 being configured to extend from a first bone (e.g., the femur 20) of a joint (e.g., the knee 10) to a second bone (e.g., the tibia 30) of the joint. In some embodiments, the graft 90 may be a generally flat member that is folded over at a first end and/or a second end to form a multi-layered structure to form a multi-layered structure comprising a first portion (e.g., first portion 96; FIG. 13) and a second portion (e.g., second portion 98; FIG. 13). The graft 90 may include one or more filaments 91 wrapped around the graft 90 and/or the multi-layered structure to secure the graft 90 to itself and/or to secure the multi-layered structure together. For example, the one or more filaments 91 may secure the first portion and the second portion of the graft 90 to each other, against each other, in intimate contact with each other, etc. Additional structure and/or elements may be added to further secure and/or bond the multi-layered structure together, where desired. For example, an adhesive and/or a bonding agent may be applied to the graft 90 and/or the multi-layered structure. In at least some embodiments, the bioactive scaffold and/or the tissue matrix 400 may surround the graft 90 before the graft 90 is implanted into the joint. In some embodiments, the graft 90 may include a first tether 440 extending axially from the graft 90. The first tether 440 may be secured to the first end of the graft 90. In some embodiments, the first tether 440 may be fixedly secured to the first end of the graft 90. In some embodiments, the first tether 440 may pass between layers of the multi-layered structure and/or under the fold formed at the first end of the graft 90. The first tether 440 may be configured to secure the graft 90 to the first bone 20, using an anchoring member or other securement element, for example.

In some embodiments, the tissue matrix 400 may include and/or be formed from a biocompatible material such as collagen. Preferably, the tissue matrix 400 includes collagen type I, although other forms of collagen (e.g., types II, III, IV, V, IX, or X) may be used and/or may be included and/or mixed with collagen type I. In some embodiments, the tissue matrix 400 may have a thickness of about 5 millimeters or less, about 4 millimeters or less, about 3 millimeters or less, about 2 millimeters or less, or another suitable thickness. Preferably, the tissue matrix 400 has a thickness of about 3 millimeters or less. In some embodiments, the bioactive scaffold and/or the tissue matrix 400 may include bioactive components configured to stimulate a healing response within the patient. In some embodiments, the composition of the bioactive scaffold and/or the tissue matrix 400 may be varied and/or optimized to promote a desired healing response in each anatomical portion of the graft 90. In some embodiments, the bioactive scaffold and/or the tissue matrix 400 may be coated and/or impregnated with a therapeutic substance configured to promote or inhibit a particular response from the patient, the graft 90, and/or the tissue matrix 400.

In some embodiments, the tissue matrix 400 may include a first orifice 410 formed therein proximate the first end of the graft 90. The first tether 440 may be configured to extend axially away from the first end of the graft 90 and through the first orifice 410. In at least some embodiments, a cross-sectional area of the first orifice 410 may be less than a cross-sectional area of the graft 90 proximate and/or adjacent the first end of the graft 90, as seen in FIG. 11. Accordingly, in at least some embodiments, the graft 90 is unable to pass through the first orifice 410 formed in the tissue matrix 400. In some embodiments, the cross-sectional area of the first orifice 410 is at least twice as great as a cross-sectional area of the first tether 440.

In some embodiments, the first tether 440 may be configured to extend into and/or through the first hole 22 formed in the first bone 20 to a first anchoring member 450. In some embodiments, the first anchoring member 450 may be a disc, a plate, or other element configured to span the first hole 22. An outer perimeter of the first anchoring member 450 may be substantially round, oblong, ovoid, elliptical, rectangular, or other suitable shape. In some embodiments, the first anchoring member 450 may include at least one hole formed therein, wherein the at least one hole is configured to receive the first tether 440 therethrough. In at least some embodiments, the first anchoring member 450 may be configured to rest against an outside surface of the first bone 20 when engaged with the first tether 440, as seen in FIG. 12. In some embodiments, an outer extent of the first anchoring member 450 may be greater than an outer extent and/or a cross-sectional area of the first hole 22 formed in the first bone 20. In some embodiments, the first anchoring member 450 may be substantially rigid and/or inflexible. For example, the first anchoring member 450 may be formed from a metallic material, a polymeric material, and/or a composite or reinforced material. As such, the first anchoring member 450 cannot be pulled into or through the first hole 22 formed in the first bone 20.

In some embodiments, the tissue matrix 400 and/or the graft 90 may be configured to span a gap between the first bone 20 and the second bone 30. In some embodiments, a second end of the graft 90 may be secured to the second bone (e.g., the tibia 30) of the joint (e.g., the knee 10) using one or more securement means known in the art. In one example, the second end of the graft 90 may be secured to the second bone using the second anchor member 94 (e.g., a bone screw, a nail, etc.). In another example, the second end of the graft 90 may be secured to the second bone using an adhesive or bonding agent. In yet another example, the second end of the graft 90 may be secured to the second bone using a second anchoring member and a second tether similar in form and function to the first anchoring member 450 and the first tether 440 described above. In some embodiments, the tissue matrix 400 and/or the graft 90 may be configured to extend into the second hole 32 formed in the second bone 30. In some embodiments, the second end of the graft 90 may be secured within the second hole 32 formed in the second bone 30.

In another alternative embodiment illustrated in FIG. 13, a bioactive scaffold for use in reconstruction of an anterior cruciate ligament of a patient may comprise a tissue matrix 500 configured to surround a graft 90, the graft 90 being configured to extend from a first bone (e.g., the femur 20) of a joint (e.g., the knee 10) to a second bone (e.g., the tibia 30) of the joint. In some embodiments, the graft 90 may be a generally flat member that is folded over at a first end and/or a second end to form a multi-layered structure comprising a first portion 96 and a second portion 98. The graft 90 may include one or more filaments 91 wrapped around the graft 90 and/or the multi-layered structure to secure the graft 90 to itself and/or to secure the multi-layered structure together. For example, the one or more filaments 91 may secure the first portion 96 and the second portion 98 of the graft 90 to each other, against each other, in intimate contact with each other, etc. Additional structure and/or elements may be added to further secure and/or bond the multi-layered structure together, where desired. For example, an adhesive and/or a bonding agent may be applied to the graft 90 and/or the multi-layered structure.

In some embodiments, the bioactive scaffold and/or the tissue matrix 500 may be a substantially flat structure, such as a strip. The bioactive scaffold and/or the tissue matrix 500 maybe configured to drape over the graft 90, as seen in FIG. 13, such that the bioactive scaffold and/or the tissue matrix 500 may be draped and/or folded over the first end of the graft 90 to form a first portion 502 and a second portion 504 disposed opposite the first portion 502 about and/or with respect to the graft 90. In some embodiments, the first portion 96 of the graft 90 may be disposed adjacent the first portion 502 of the bioactive scaffold and/or the tissue matrix 500, and the second portion 98 of the graft 90 may be disposed adjacent the second portion 504 of the bioactive scaffold and/or the tissue matrix 500. In some embodiments, a lateral portion of the graft 90 may be exposed between the first portion 502 and the second portion 504 of the bioactive scaffold and/or the tissue matrix 500. For example, the bioactive scaffold and/or the tissue matrix 500 may not completely surround the graft 90.

In at least some embodiments, the bioactive scaffold and/or the tissue matrix 500 may be draped over the graft 90 before the graft 90 is implanted into the joint. In some embodiments, the graft 90 may include a first tether 540 extending axially from the graft 90. The first tether 540 may be secured to the first end of the graft 90. In some embodiments, the first tether 540 may be fixedly secured to the first end of the graft 90. In some embodiments, the first tether 540 may pass between layers of the multi-layered structure and/or under the fold formed at the first end of the graft 90. The first tether 540 may be configured to secure the graft 90 to the first bone 20, using an anchoring member or other securement element, for example.

In some embodiments, the tissue matrix 500 may include and/or be formed from a biocompatible material such as collagen. Preferably, the tissue matrix 500 includes collagen type I, although other forms of collagen (e.g., types II, III, IV, V, IX, or X) may be used and/or may be included and/or mixed with collagen type I. In some embodiments, the tissue matrix 500 may have a thickness of about 5 millimeters or less, about 4 millimeters or less, about 3 millimeters or less, about 2 millimeters or less, or another suitable thickness. Preferably, the tissue matrix 500 has a thickness of about 3 millimeters or less. In some embodiments, the bioactive scaffold and/or the tissue matrix 500 may include bioactive components configured to stimulate a healing response within the patient. In some embodiments, the composition of the bioactive scaffold and/or the tissue matrix 500 may be varied and/or optimized to promote a desired healing response in each anatomical portion of the graft 90. In some embodiments, the bioactive scaffold and/or the tissue matrix 500 may be coated and/or impregnated with a therapeutic substance configured to promote or inhibit a particular response from the patient, the graft 90, and/or the tissue matrix 500.

The first tether 540 may be configured to extend axially away from the first end of the graft 90. In some embodiments, the first tether 540 may be configured to extend into and/or through the first hole 22 formed in the first bone 20 to a first anchoring member, such as the first anchoring member 450 described herein. In some embodiments, the first anchoring member 450 may be a disc, a plate, or other element configured to span the first hole 22. An outer perimeter of the first anchoring member 450 may be substantially round, oblong, ovoid, elliptical, rectangular, or other suitable shape. In some embodiments, the first anchoring member 450 may include at least one hole formed therein, wherein the at least one hole is configured to receive the first tether 540 therethrough. In at least some embodiments, the first anchoring member 450 may be configured to rest against an outside surface of the first bone 20 when engaged with the first tether 540, similar to the configuration shown in FIG. 12. In some embodiments, an outer extent of the first anchoring member 450 may be greater than an outer extent and/or a cross-sectional area of the first hole 22 formed in the first bone 20. In some embodiments, the first anchoring member 450 may be substantially rigid and/or inflexible. For example, the first anchoring member 450 may be formed from a metallic material, a polymeric material, and/or a composite or reinforced material. As such, the first anchoring member 450 cannot be pulled into or through the first hole 22 formed in the first bone 20.

In some embodiments, the tissue matrix 500 and/or the graft 90 may be configured to span a gap between the first bone 20 and the second bone 30. In some embodiments, a second end of the graft 90 may be secured to the second bone (e.g., the tibia 30) of the joint (e.g., the knee 10) using one or more securement means known in the art. In one example, the second end of the graft 90 may be secured to the second bone using the second anchor member 94 (e.g., a bone screw, a nail, etc.). In another example, the second end of the graft 90 may be secured to the second bone using an adhesive or bonding agent. In yet another example, the second end of the graft 90 may be secured to the second bone using a second anchoring member and a second tether similar in form and function to the first anchoring member 450 and the first tether 540 described above. In some embodiments, the tissue matrix 500 and/or the graft 90 may be configured to extend into the second hole 32 formed in the second bone 30. In some embodiments, the second end of the graft 90 may be secured within the second hole 32 formed in the second bone 30.

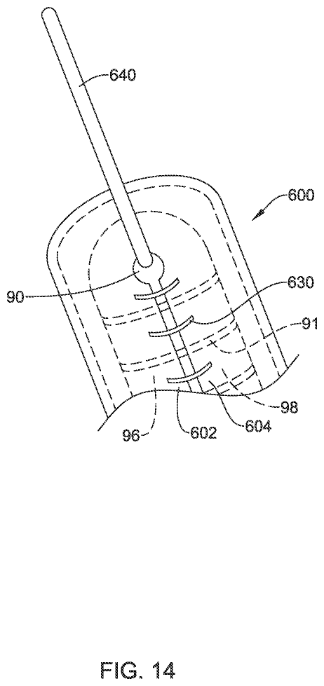

In another alternative embodiment illustrated in FIG. 14, a bioactive scaffold for use in reconstruction of an anterior cruciate ligament of a patient may comprise a tissue matrix 600 configured to surround a graft 90, the graft 90 being configured to extend from a first bone (e.g., the femur 20) of a joint (e.g., the knee 10) to a second bone (e.g., the tibia 30) of the joint. In some embodiments, the graft 90 may be a generally flat member that is folded over at a first end and/or a second end to form a multi-layered structure comprising a first portion 96 and a second portion 98. The graft 90 may include one or more filaments 91 wrapped around the graft 90 and/or the multi-layered structure to secure the graft 90 to itself and/or to secure the multi-layered structure together. For example, the one or more filaments 91 may secure the first portion 96 and the second portion 98 of the graft 90 to each other, against each other, in intimate contact with each other, etc. Additional structure and/or elements may be added to further secure and/or bond the multi-layered structure together, where desired. For example, an adhesive and/or a bonding agent may be applied to the graft 90 and/or the multi-layered structure.

In some embodiments, the bioactive scaffold and/or the tissue matrix 600 may be a substantially flat structure, such as a strip. The bioactive scaffold and/or the tissue matrix 600 maybe configured to drape over the graft 90, as seen in FIG. 14, such that the bioactive scaffold and/or the tissue matrix 600 may be draped and/or folded over the first end of the graft 90 to form a first portion 602 and a second portion 604 disposed opposite the first portion 602 about and/or with respect to the graft 90. In some embodiments, the first portion 96 of the graft 90 may be disposed adjacent the first portion 602 of the bioactive scaffold and/or the tissue matrix 600, and the second portion 98 of the graft 90 may be disposed adjacent the second portion 604 of the bioactive scaffold and/or the tissue matrix 600. In some embodiments, the first portion 602 and the second portion 604 of the bioactive scaffold and/or the tissue matrix 600 may be secured to each other along lateral seams by a plurality of filaments 630. The lateral seams may be formed by bring edges of the first portion 602 and the second portion 604 together. In some embodiments, the edges of the first portion 602 and the second portion 604 may abut each other. In some embodiments, the edges of the first portion 602 and the second portion 604 may overlap each other. In some embodiments, the edges of the first portion 602 and the second portion 604 may remain spaced apart from each other after securing the first portion 602 to the second portion 604 along the lateral seams. In some embodiments, after securing the first portion 602 to the second portion 604 along the lateral seams, the bioactive scaffold and/or the tissue matrix 600 may substantially surround the graft 90. Other configurations are also contemplated.

In at least some embodiments, the bioactive scaffold and/or the tissue matrix 600 may be draped over the graft 90 before the graft 90 is implanted into the joint. In some embodiments, the graft 90 may include a first tether 640 extending axially from the graft 90. The first tether 640 may be secured to the first end of the graft 90. In some embodiments, the first tether 640 may be fixedly secured to the first end of the graft 90. In some embodiments, the first tether 640 may pass between layers of the multi-layered structure and/or under the fold formed at the first end of the graft 90. The first tether 640 may be configured to secure the graft 90 to the first bone 20, using an anchoring member or other securement element, for example.

In some embodiments, the tissue matrix 600 may include and/or be formed from a biocompatible material such as collagen. Preferably, the tissue matrix 600 includes collagen type I, although other forms of collagen (e.g., types II, III, IV, V, IX, or X) may be used and/or may be included and/or mixed with collagen type I. In some embodiments, the tissue matrix 600 may have a thickness of about 5 millimeters or less, about 4 millimeters or less, about 3 millimeters or less, about 2 millimeters or less, or another suitable thickness. Preferably, the tissue matrix 600 has a thickness of about 3 millimeters or less. In some embodiments, the bioactive scaffold and/or the tissue matrix 600 may include bioactive components configured to stimulate a healing response within the patient. In some embodiments, the composition of the bioactive scaffold and/or the tissue matrix 600 may be varied and/or optimized to promote a desired healing response in each anatomical portion of the graft 90. In some embodiments, the bioactive scaffold and/or the tissue matrix 600 may be coated and/or impregnated with a therapeutic substance configured to promote or inhibit a particular response from the patient, the graft 90, and/or the tissue matrix 600.

The first tether 640 may be configured to extend axially away from the first end of the graft 90. In some embodiments, the first tether 640 may be configured to extend into and/or through the first hole 22 formed in the first bone 20 to a first anchoring member, such as the first anchoring member 450 described herein. In some embodiments, the first anchoring member 450 may be a disc, a plate, or other element configured to span the first hole 22. An outer perimeter of the first anchoring member 450 may be substantially round, oblong, ovoid, elliptical, rectangular, or other suitable shape. In some embodiments, the first anchoring member 450 may include at least one hole formed therein, wherein the at least one hole is configured to receive the first tether 640 therethrough. In at least some embodiments, the first anchoring member 450 may be configured to rest against an outside surface of the first bone 20 when engaged with the first tether 640, similar to the configuration shown in FIG. 12. In some embodiments, an outer extent of the first anchoring member 450 may be greater than an outer extent and/or a cross-sectional area of the first hole 22 formed in the first bone 20. In some embodiments, the first anchoring member 450 may be substantially rigid and/or inflexible. For example, the first anchoring member 450 may be formed from a metallic material, a polymeric material, and/or a composite or reinforced material. As such, the first anchoring member 450 cannot be pulled into or through the first hole 22 formed in the first bone 20.

In some embodiments, the tissue matrix 600 and/or the graft 90 may be configured to span a gap between the first bone 20 and the second bone 30. In some embodiments, a second end of the graft 90 may be secured to the second bone (e.g., the tibia 30) of the joint (e.g., the knee 10) using one or more securement means known in the art. In one example, the second end of the graft 90 may be secured to the second bone using the second anchor member 94 (e.g., a bone screw, a nail, etc.). In another example, the second end of the graft 90 may be secured to the second bone using an adhesive or bonding agent. In yet another example, the second end of the graft 90 may be secured to the second bone using a second anchoring member and a second tether similar in form and function to the first anchoring member 450 and the first tether 640 described above. In some embodiments, the tissue matrix 600 and/or the graft 90 may be configured to extend into the second hole 32 formed in the second bone 30. In some embodiments, the second end of the graft 90 may be secured within the second hole 32 formed in the second bone 30.

In use, after implantation of the graft 90 and the tissue matrix (e.g., ref. 100, 200, 300, 400) within the joint (e.g., the knee 10), the patient's body may proceed to remodel the tissue of the graft 90 from its original tissue type (e.g., tendon, etc.) into the type of tissue it is intended to replace (e.g., ligament). The rehabilitation process can be long (e.g., 9-12 months), and during the rehabilitation process, the graft 90 may weaken as the tissue is remodeled by the patient's body, thereby placing the graft 90 at risk of injury (e.g., rupture). When a tissue is moved (e.g., when a tendon is used to replace a ligament), the original tissue breaks down before it becomes strong enough to function again via remodeling. During remodeling of the tissue, the original cells of the tissue die off and must be replaced by new ones.

The tissue matrix according to the current disclosure may shorten the length of time of the rehabilitation process, may accelerate remodeling of tissue, and/or may increase final strength of the graft 90 after remodeling. During rehabilitation, the tissue matrix may facilitate cellular attachment and/or ingrowth as the graft 90 is remodeled into the desired tissue type by providing more locations for new cells to attach and infiltrate into the graft 90. Repopulation of the graft 90 by external cells is a necessary process for healing which to may be facilitated by the tissue matrix since it provides additional cell attachment locations and may attract the desired and/or requisite cells. In some cases, the tissue matrix may also act as a barrier to exclude undesirable cytokines which may impede healing and/or remodeling of the graft 90.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A bioactive scaffold for use in reconstruction of an anterior cruciate ligament of a patient, comprising:
a tissue matrix configured to wrap around a graft, the graft being configured to extend from a first bone of a joint to a second bone of the joint;
wherein the tissue matrix includes collagen type I;
wherein the tissue matrix includes a first plurality of apertures extending through the tissue matrix along a first edge of the tissue matrix and a second plurality of apertures extending through the tissue matrix along a second edge of the tissue matrix; and
at least one filament configured to be laced through the first plurality of apertures and the second plurality of apertures and draw the first edge of the tissue matrix toward the second edge of the tissue matrix when the at least one filament is subjected to tension thereby closing the tissue matrix around the graft.

2. The bioactive scaffold of claim 1, wherein after the at least one filament is subjected to tension to close the tissue matrix around the graft, the tissue matrix applies a compressive force to the graft.

3. The bioactive scaffold of claim 1, wherein the first edge of the tissue matrix is configured to abut the second edge of the tissue matrix when the at least one filament is subjected to tension to close the tissue matrix around the graft.

4. The bioactive scaffold of claim 1, wherein the first edge of the tissue matrix is configured to circumferentially overlap the second edge of the tissue matrix when the at least one filament is subjected to tension to close the tissue matrix around the graft.

5. The bioactive scaffold of claim 4, wherein the first plurality of apertures is aligned with the second plurality of apertures.

6. The bioactive scaffold of claim 4, wherein the first plurality of apertures is disposed opposite the second plurality of apertures relative to the first edge of the tissue matrix.

7. The bioactive scaffold of claim 1, wherein the at least one filament is configured to be laced through the first plurality of apertures and the second plurality of apertures in an alternating fashion.

8. The bioactive scaffold of claim 1, wherein a first portion of the graft is configured to extend into a first hole formed in the first bone.

9. The bioactive scaffold of claim 8, wherein a first portion of the tissue matrix is configured to extend into the first hole formed in the first bone.

10. A bioactive scaffold for use in reconstruction of an anterior cruciate ligament of a patient, comprising:
a tissue matrix configured to wrap around a graft, the graft being configured to extend from a first bone of a joint to a second bone of the joint;
wherein the tissue matrix includes collagen type I;
wherein the tissue matrix is wrapped helically around the graft.

11. The bioactive scaffold of claim 10, wherein after wrapping the tissue matrix around the graft, the tissue matrix is in direct contact with the graft from a first location adjacent the first bone to a second location adjacent the second bone.

12. The bioactive scaffold of claim 10, wherein a first edge of the tissue matrix is configured to abut a second edge of the tissue matrix after wrapping the tissue matrix around the graft.

13. The bioactive scaffold of claim 10, wherein a first edge of the tissue matrix is configured to circumferentially overlap a second edge of the tissue matrix after wrapping the tissue matrix around the graft.

14. The bioactive scaffold of claim 10, wherein the tissue matrix is configured to retain its shape and position relative to the graft after being helically wrapped around the graft.

15. A bioactive scaffold for use in reconstruction of an anterior cruciate ligament of a patient, comprising:
a tissue matrix configured to surround a graft, the graft being configured to extend from a first bone of a joint to a second bone of the joint;
wherein the graft includes a first tether extending axially from the graft, the first tether being configured to secure the graft to the first bone;
wherein the tissue matrix includes collagen type I;
wherein the tissue matrix includes a first orifice proximate a first end of the graft, the first tether extending axially through the first orifice;
wherein a cross-sectional area of the first orifice is less than a cross-sectional area of the graft proximate the first end of the graft;
wherein the cross-sectional area of the first orifice is at least twice as great as a cross-sectional area of the first tether; and
wherein the tissue matrix is configured to span a gap between the first bone and the second bone.

16. The bioactive scaffold of claim 15, wherein a first edge of the tissue matrix is configured to abut a second edge of the tissue matrix after wrapping the tissue matrix around the graft.

17. The bioactive scaffold of claim 15, wherein a first edge of the tissue matrix is configured to circumferentially overlap a second edge of the tissue matrix after wrapping the tissue matrix around the graft.
